(19) United States
(12) Patent Application Publication
(10) Pub. No.: US 2006/0241462 A1
(43) Pub. Date: Oct. 26, 2006

(54) METHOD OF INTRACRANIAL ULTRASOUND IMAGING AND RELATED SYSTEM

(76) Inventors: Yi-Hong Chou, Taipei City (TW); Chung-Yuo Wu, Taipei Hsien (TW); Ta-Jung Su, Taipei Hsien (TW); Meng-Tsung Lo, Taipei Hsien (TW)

Correspondence Address: NORTH AMERICA INTELLECTUAL PROPERTY CORPORATION, P.O. BOX 506, MERRIFIELD, VA, 22116, US

(21) Appl. No.: 10/906,969

(22) Filed: Mar. 14, 2005

(51) Int. Cl. A61B 8/00

(52) U.S. Cl. 600/455

(57) ABSTRACT

A method of intracranial ultrasound imaging applied in detecting a cranial blood vessel having blood filled with micro-bubbles formed by an injected contrast agent and generating blood vessel images includes: (1) emitting a plurality of ultrasound signals having bandwidths to the cranial blood vessel in sequence, (2) receiving an echoed signal from a micro-bubble, (3) performing a spectral analysis on the echoed signal and extracting a low-frequency response, the bandwidth of the low-frequency response similar to the bandwidth of the ultrasound signal, and (4) calculating a location and a depth of the micro-bubble in the cranium according to the low-frequency response and generating a corresponding blood vessel image.

### BACKGROUND OF INVENTION

1. Field of the Invention

The present invention relates to a method of intracranial ultrasound imaging, and more particularly, to a method of intracranial ultrasound imaging by using an ultrasound contrast agent and a specific signal process.

2. Description of the Prior Art

Medical ultrasound instruments have the advantages of safety and low price. Therefore, technology for ultrasound imaging has been widely used in clinical diagnosis, such as heart, abdominal region, and gynecological examinations.

However, ultrasound signals are dramatically attenuated when transmitted through the cranium surrounding the brain. Thus, ultrasound imaging technology is mostly applied in detecting whether the linear structure shifts or a ventricle expands inside the cranium, and is seldom applied in diagnosis of brain diseases, such as brain hematoma, arteriovenous aneurysm, arteriovenous malformation, etc. Usually, expensive instruments, such as computer axial tomography (CAT) and nuclear magnetic resonance (NMR) devices, are used to take intracranial images. The following describes improvements and studies for intracranial ultrasound imaging.

One way is to examine specific parts, such as a newborn's unclosed fontanel, an adult's foramen magnum, or an eyehole, by a wide-angle sector scanner. However, ultrasound signals and imaging are thus confined to such specific parts, and many areas inside the cranium cannot be detected, especially for children over one year old or adults.

Secondly, scan converters with higher resolution and transducers with higher frequency have been developed recently. The resolution of ultrasound images is improved by the technology of emitting and receiving signals by high frequency. However, the above technology and tools still cannot overcome the attenuation issue when a high-frequency ultrasound signal travels through the cranium, and thereby images of heart-related diseases or images inside the cranium cannot be obtained clearly. The prior art still utilizes low-frequency signals to reduce the attenuation, but the image resolution is extremely poor.

Thirdly, in order to receive signals from the blood stream, an injection of contrast agent into blood or lymph has been used. Micro-bubbles of such a contrast agent are helpful in creating better acoustic wave feedback. Therefore, the purpose of signal improvement is achieved, which assists in measuring related parameters.

Please refer to FIG. 1, which is a frequency spectrum of ultrasound echoed signals associated with the contrast agent. As shown in FIG. 1, there are a fundamental response 11, a second harmonic response 12, and a subharmonic response 13. The latter two are non-linear responses and require higher emitting sound pressure to stimulate micro-bubbles, wherein the sound pressure required by the subharmonic response 13 is the highest.

The fundamental response 11 can be found in blood-flow and peripheral tissue, and thereby the fundamental response 11 cannot be used for comparison and recognition.

For one thing, after the second harmonic response 12 travels through the cranium, the second harmonic response 12 is dramatically attenuated due to its high frequency. Additionally, the second harmonic response 12 also occurs in mammal tissues. So it is difficult to use the second harmonic response 12 to distinguish between blood, lymph, and peripheral tissue.

The obvious subharmonic response 13 is excited by high pressure, which causes micro-bubbles to break more easily. If the method is used for intracranial imaging, micro-bubbles breaking might be a threat to the brain.

### SUMMARY OF INVENTION

It is therefore a primary objective of the claimed invention to provide a method of intracranial ultrasound imaging not confined to specific parts and related system to solve the above-mentioned problem.

The claimed invention provides a method and system to efficiently obtain signals from the blood stream inside the cranium and generate images.

The claimed invention also provides a precise and safe method and system to generate intracranial ultrasound images.

The claimed invention can detect a cranial blood vessel having blood filled with micro-bubbles formed by a contrast agent, and generate images. The system includes an ultrasound transducer, a transmitter module connected to the ultrasound transducer, a receiver module, and a signal processing module.

The method of the claimed invention includes:

(1) The transmitter module generates a driving signal to drive the ultrasound transducer to emit a plurality of ultrasound signals having bandwidths, each of which is a short pulse, to a cranial blood vessel. The attenuation of signals analyzed by the claimed invention is slight. Therefore, the ultrasound transducer can emit ultrasound signals from any intracranial areas to travel through cranial bones into the cranial blood vessel.

(2) The ultrasound transducer senses an echoed signal from micro-bubbles and conveys the echoed signal to the receiver module.

(3) The signal processing module receives the echoed signal and performs a spectral analysis on the echoed signal to obtain a fundamental response, a second harmonic response, a subharmonic response, and a low-frequency response. The low-frequency response occurs at a part close to a DC component of the frequency spectrum. The bandwidth of the low-frequency response is similar to the bandwidth of the fundamental response or the bandwidth of the emitted signal. The generation of the low-frequency response can be supported by the theory and experimental results of the claimed invention. When micro-bubbles are excited by the dual-frequency acoustic signal with its two frequencies (of suitable transmission bandwidth) being close enough. A difference between the two frequencies, which is close to a DC component of the frequency spectrum (i.e., the low-frequency response) will be excited to form the low-frequency response.

(4) The signal processing module uses a band-pass filter to capture the low-frequency response to avoid disturbance from direct current and fundamental frequency. This step only retains signals from the blood vessel and it is very helpful for generating images.

(5) The location and depth of micro-bubbles in the cranium are calculated based on the low-frequency response, and blood vessel images are generated accordingly.

The entire quality of ultrasound images depends on emitted and received signals. The prior art cannot overcome the attenuation issue when signals travel through the cranium, and thereby low-frequency signals are emitted and received to get low-resolution images. However, the claimed invention can improve the above issue. The claimed invention emits high-frequency signals and receives the low-frequency response from the echoed signal of the micro-bubble. Although the attenuation occurs when signals are emitted, the low-frequency response of the echoed signal is attenuated quite less than the high-frequency signal. Therefore, the claimed invention emits signals having proper strength, or adds proper strength to signals when signals are emitted, such that the low-frequency response can be generated after the ultrasound signal travels through the cranium, and clear blood vessel images can be generated. In other words, the claimed invention adopts the method of emitting high-frequency signals and receiving low-frequency signals instead of emitting and receiving low-frequency signals to improve the resolution of images.

The claimed invention adopts the low-frequency response, which is not excited by high pressure as the subharmonic response, and thereby there is no micro-bubble break issue. Compared to the prior art, the claimed invention provides a safer method of intracranial ultrasound imaging. The claimed invention can not only generate images of human craniums, but also can be applied in other organs of mammals, especially organs obstructed by bone. Also, the claimed invention can be implemented in crack detection in steelwork. The claimed invention overcomes the dramatic attenuation when ultrasound signals travel through media having high attenuation factors, which results in images with low resolution.

These and other objectives of the present invention will no doubt become obvious to those of ordinary skill in the art after reading the following detailed description of the preferred embodiment that is illustrated in the various figures and drawings.

### BRIEF DESCRIPTION OF DRAWINGS

FIG. 1 is a frequency spectrum of echoed ultrasound signals in the prior art.

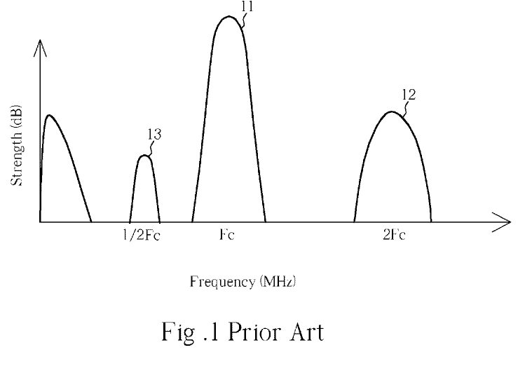

FIG. 2 is a diagram of an intracranial ultrasound imaging system based on the present invention.

FIG. 3 is a flowchart of the intracranial ultrasound imaging system based on the present invention.

### DETAILED DESCRIPTION

Regarding the above description and detailed technology of the present invention, a best embodiment with drawings are disclosed as follows.

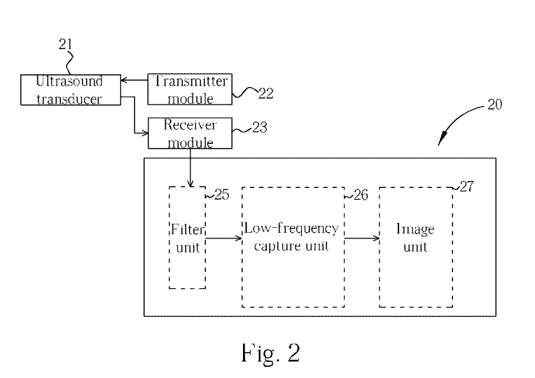

Shown in FIG. 2 is a diagram of an intracranial ultrasound imaging system implemented in an ultrasound system. To shed light on the present invention, the best embodiment detects a cranial blood vessel and generates a corresponding image. The contrast agent is injected into the cranial blood vessel by intravenous injection in advance so that the blood of the cranial blood vessel has a lot of micro-bubbles. The system comprises an ultrasound transducer 21, a transmitter module 22, a receiver module 23, and a signal processing module 20. The signal processing module 20 includes a filter unit 25, a low-frequency capture unit 26, and an image unit 27.

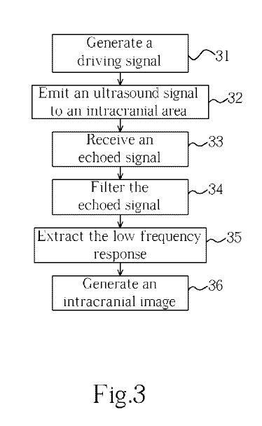

Please refer to FIG. 3, which is a flowchart of intracranial ultrasound imaging. The steps are as follows.

Step 31: The transmitter module 22 generates a driving signal to the ultrasound transducer 21. The ultrasound transducer 21 is nestled anywhere on the patient's head in advance.

Step 32: According to the driving signal, the ultrasound transducer 21 emits a plurality of ultrasound signals having bandwidths to the cranial blood vessel. Due to the quality of ultrasound images depending on emitted and received signals, the present invention emits high-frequency signals to obtain a better resolution of images. In addition, the present invention adds strength compensation to the emitted signal so that the energy is enough to generate the low-frequency response after traveling through the cranium, and clear images can be generated. The strength compensation is calculated according to the product of the strength of the emitted signal and an attenuation factor. Generally, the attenuation factor is 1.3 dB/cm.MHz

Step 33: The ultrasound transducer 21 receives an echoed signal from a micro-bubble and conveys the echoed signal to the receiver module 23.

Step 34: The receiver module 23 conveys the echoed signal to the filter unit 25 of the signal processing module 20 to filter the echoed signal so as to improve the quality of the detected echoed signal.

Step 35: The low-frequency capture unit 26 receives the echoed signal from the filter unit 25, and performs a spectral analysis on the echoed signal. According to the frequency distribution of the echoed signal, a fundamental response 41 whose central frequency and bandwidth are quite similar to those of the ultrasound signal is obtained, and a low-frequency response 42, which is close to a DC component, is obtained. Then a band-pass filter is used to extract the low-frequency response 42. The bandwidth of the low-frequency response 42 is similar to that of the fundamental response 41. This step only retains signals from the blood vessel and it is very helpful for generating images.

Step 36: The location and depth of micro-bubbles in the cranium are calculated based on the low-frequency response, and images of the blood vessel are generated accordingly.

To sum up, although the attenuation occurs when signals are emitted, the low-frequency response of the echoed signal from the micro-bubbles is attenuated significantly less than the high-frequency signal, and is taken as patterns to generate images. Therefore, the claimed invention only needs to add proper strength to signals when signals are emitted, such that clear blood vessel images can be generated. Thus, the present invention can provide safe, economical, and precise imaging, and the imaging locations are not limited to specific parts.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method of intracranial ultrasound imaging applied in detecting a cranial blood vessel having blood filled with micro-bubbles formed by an injected contrast agent and generating blood vessel images, the method comprising:
(a) emitting a plurality of ultrasound signals having bandwidths to the cranial blood vessel in sequence;
(b) receiving an echoed signal from a micro-bubble inside the cranial blood vessel;
(c) performing a spectral analysis on the echoed signal and extracting a low-frequency response, the bandwidth of the low-frequency response being similar to the bandwidth of the ultrasound signal; and
(d) calculating a location and a depth of the micro-bubble in the cranium according to the low-frequency response and generating a corresponding blood vessel image.

2. The method of claim 1 wherein the strength of the emitted signal in step (a) is adjusted according to an attenuation factor.

3. A system of intracranial ultrasound imaging applied in detecting a cranial blood vessel having blood filled with micro-bubbles formed by an injected contrast agent and generating blood vessel images, the system comprising:
a transmitter module for emitting a plurality of ultrasound signals having bandwidths to the cranial blood vessel in sequence;
a receiver module for receiving an echoed signal from a micro-bubble; and
a signal processing module comprising:
a low-frequency capture unit for performing a spectral analysis on the echoed signal and extracting a low-frequency response, the bandwidth of the low-frequency response being similar to the bandwidth of the ultrasound signal; and
an image unit for calculating a location and a depth of the micro-bubble in the cranium according to the low-frequency response of the echoed signal, and generating a corresponding blood vessel image accordingly.

4. The system of claim 3 wherein the strength of the emitted signal is adjusted according to an attenuation factor.
